(19) United States
(12) Patent Application Publication
(10) Pub. No.: US 2019/0307359 A1
(43) Pub. Date: Oct. 10, 2019

(54) COMPLIANT SENSING SYSTEM APPLICABLE FOR PALPATION

(71) Applicants: University of Maryland, College Park, College Park, MD (US); University of Illinois at Urbana-Champaign, Urbana, IL (US)

(72) Inventors: Elisabeth Smela, Silver Spring, MD (US); Miao Yu, Potomac, MD (US); Hugh A. Bruck, Wheaton, MD (US); Thenkurussi Kesavadas, Urbana, IL (US)

(73) Assignees: University of Maryland, College Park, College Park, MD (US); University of Illinois at Urbana-Champaign, Urbana, IL (US)

(21) Appl. No.: 16/421,990

(22) Filed: May 24, 2019

Related U.S. Application Data
(63) Continuation-in-part of application No. 16/112,609, filed on Aug. 24, 2018.
(60) Provisional application No. 62/549,672, filed on Aug. 24, 2017.

(51) Int. Cl. A61B 5/053; A61B 5/00

(52) U.S. Cl. CPC A61B 5/0536; A61B 5/0053; A61B 5/6804; A61B 5/6812; A61B 2562/0261; A61B 5/6843; A61B 5/4312; A61B 5/4387; A61B 5/7425

(57) ABSTRACT

A tactile sensing system includes at least a stretchable strain sensing layer, an inflatable reservoir, and a means for detecting strain in the stretchable strain sensing layer. The tactile sensing layer may be configured as a tumor detection system by configuring the inflatable reservoir to apply pressure to at least part of a tissue in conjunction with an anatomical contact structure and the stretchable strain sensing layer to be in contact with a region of the surface of the tissue.

### CROSS-REFERENCE TO RELATED APPLICATIONS

This application is a continuation-in-part of U.S. patent application Ser. No. 16/112,609 filed on Aug. 24, 2018, claiming the benefit of, and priority to, U.S. Provisional Patent Application No. 62/549,672 filed on Aug. 24, 2017, the entire contents of each of which are incorporated herein by reference.

### STATEMENT OF GOVERNMENT SUPPORT

This invention was made with U.S. government support under IIS1317913 awarded by NSF. The U.S. government has certain rights in the invention.

### BACKGROUND

### 1. Technical Field

The present disclosure relates to tactile sensing systems and more particularly to tactile sensing systems that include, but are not limited to, applications for medical sensing.

### 2. Discussion of Related Art

Health care providers can use touch to determine the size, texture, and location of a tumor. As part of a clinical examination to screen for cancer, a physician or other trained health practitioner performs a manual palpation. Palpation can detect malignant masses because they are generally harder than the surrounding tissue.

Breast cancer, prostate cancer, and other types of cancer (e.g., of the throat and tongue) can be detected by manual palpation.

Mammography is a conventional technique for imaging and tumor detection that requires a sophisticated, expensive medical facility operated by trained medical personnel.

However, in parts of the world with an insufficient number of medical personnel who are properly trained in palpation or imaging technologies for detecting breast cancer, or in parts of the world where there are too few medical personnel to screen a large number of patients, or in parts of the world where health providers are unfamiliar with breast cancer, or for populations in which individuals are reluctant to visit medical clinics for screening, breast cancer often goes undetected in the stages when it is treatable.

The value of tactile sensing for breast cancer detection is established. Devices are known such as capacitive sensing probes which estimate the size, shape, hardness, and location of a mass and distinguish between benign and suspicious masses. Tumor detection depends on consistent manual application of force from the probe in the right places, and thus outcomes are governed by proper user operation. As a result, the probe is moved by a skilled practitioner over the breast to obtain an image.

The combination of sensors and inflatable reservoirs is known, generally for measurement of blood pressure. However, those sensors are substantially un-stretchable and are not designed, intended, utilized, or configured for location of masses within the anatomy of a patient.

Smart bras are known which are based on temperature measurements of anatomical regions due to the increased temperature of tumor cells as compared to normal tissue. Such devices require extended periods of time to detect changes in normal temperature profiles.

### SUMMARY

The embodiments of the present disclosure provide significant and non-obvious advantages over the prior art by providing a tactile sensing system comprising a stretchable strain sensing layer and an inflatable reservoir.

The embodiments of the present disclosure further provide significant and non-obvious advantages over the prior art by employing an inflatable reservoir and a larger sensing area to avoid the need for moving a relatively small device, allowing use by unskilled personnel.

The use of an inflatable reservoir in combination with a stretchable strain sensor provides unexpected and novel results. It allows the strain sensor to be stretched around a relatively rigid object that comes into contact with the system. The inflated reservoir holds the sensor flat except in the area where it is deformed, localizing the strain, and thus producing a localized signal source from the sensor. Use of an inflatable reservoir in combination with a stretchable strain sensor permits imaging on curved surfaces. Another advantage is that the extent of inflation can be controlled, either manually or electronically, permitting a series of measurements to be taken at different pressures, providing richer information about the mechanical properties of the objects with which it is in contact.

In an embodiment, the tactile sensing system may be configured as a tumor detection system. The tumor detection system according to the present disclosure allows standardized pressurization to be used to create a series of tactile images for lump characterization, which is expected to confer a degree of robustness against variability in user procedure and patient fit. Softer and stiffer objects can be distinguished. The use of one or more inflatable reservoirs in combination with one or more stretchable strain sensors allows automated probing of a complete area, without the need for skilled personnel. Another advantage is that measurements can be made quickly: it takes less than a minute to obtain images at a series of pressures.

The tumor detecting sensing system may include an anatomical support structure configured to allow the strain sensing layer to be in contact with at least one part of the body of the patient and to allow the inflatable reservoir to apply pressure to at least one part of the body to enable detection of a tumor within at least one body part by the strain sensing layer. The anatomical support structure may be configured to allow consistent placement of the tactile sensing system on a part of the body, allowing consistent information to be obtained without the need for positioning or manipulation of the sensing system on or over the patient by a skilled medical practitioner.

The tactile sensing system may include an electrical circuit to measure signals from the strain sensing layer(s). The electrical circuit may include a plurality of electrodes, wherein current is injected into a subset of the electrodes such that voltage readings obtained from others of the plurality of electrodes enable reconstruction of an image from the measured voltage readings. The circuit may include switches to allow the sites of current injection and voltage measurement to be changed during a measurement. The method of reconstructing an image may be done using electrical impedance tomography (EIT) or other methods known to those skilled in the art, such as machine learning or deep learning.

Data for machine learning or deep learning may include electrical data such as voltage, current, resistance, impedance, inductance, etc.; optical data such as light intensity or phase; or acoustical data such sound intensity or phase. Any such data that is not electrical is generally converted to electrical data and may be maintained in analog form or converted to digital form.

The electrical circuit may include circuitry enabling wireless transmission of data readings to a remote receiver location. The system may include an electronic device, such as a cell phone or laptop, to allow data or images to be transmitted to trained medical personnel in a distant location for analysis.

The tactile sensing system may comprise stretchable strain sensing layer covering one contiguous area or it may comprise an array of strips or an array of discrete elements.

The anatomical support structure may be configured as a cup of a brassiere to surround the breast of a patient to detect tumors occurring within that breast.

The anatomical support structure may be configured as a male athletic supporter to support the testicles of a male patient to detect tumors occurring within at least one testicle of the male patient.

As a result of the foregoing discussion, it can be appreciated that the present disclosure relates to a tactile sensing system that includes at least one stretchable strain sensing layer configured to enable contact with a region of an anatomical feature of a subject; at least one inflatable reservoir configured to enable application of pressure to a region of an anatomical feature of a subject; and an anatomical contact structure configured to enable at least one stretchable strain sensing layer to be in contact with a region of an anatomical feature of the subject and configured to enable at least one inflatable reservoir to apply pressure to a region of the anatomical feature of the subject.

In an aspect, at least a portion of the region to which pressure is enabled to be applied by at least one inflatable reservoir at least partially corresponds to the portion of the region with which at least one stretchable strain sensing layer is in contact.

In an aspect, the region to which pressure is enabled to be applied by at least one inflatable reservoir does not correspond to the region with which at least one stretchable strain sensing layer is in contact.

In an aspect, the tactile sensing system is configured wherein inflation of at least one inflatable reservoir to apply pressure to a region enables detection by at least one stretchable strain sensing layer of at least one mass having a stiffness different from the surrounding tissue within the anatomical portion of a subject.

In an aspect, the tactile sensing system is configured wherein inflation of at least one inflatable reservoir to apply pressure to a region enables concluding via at least one stretchable strain sensing layer of the absence of least one mass having a stiffness different than the surrounding tissue within the anatomical feature of a subject. The stiffness difference that is detectable depends on the minimum strain that the stretchable strain sensing layer can detect. A small stiffness difference may indicate the presence of a non-tumorous mass, such as a cyst, whereas a larger stiffness difference may indicate a tumor. The amplitude of the signal at a given pressure may therefore provide information about the nature of the mass.

In an aspect, at least one stretchable strain sensing layer is configured to be disposed in contact with at least one inflatable reservoir, enabling thereby: formation of an indentation in at least one inflatable reservoir and localized strain in at least one stretchable strain sensing layer around the indentation in at least one inflatable reservoir.

In an aspect, the tactile sensing system includes at least one stretchable strain sensing layer configured and disposed to enable contact with a first region of an anatomical feature of a subject; at least two inflatable reservoirs configured and disposed to enable the reservoirs to apply pressure to a second and third region of an anatomical feature of a subject, the reservoirs configured and disposed to be independently inflatable with respect to one another such that one of the reservoirs is enabled to apply an initial pressure to the second region of the anatomical feature of a subject that is greater than pressure applied to the third region of the anatomical feature of a subject by the second reservoir, the reservoirs configured and disposed such that the second reservoir is enabled to apply an initial pressure to the third region of the anatomical feature of a subject following or during deflation of the initial pressure applied to the second region of the anatomical feature of a subject by the first reservoir, enabling thereby detection by the stretchable strain sensing layer of at least one mass having a stiffness different from than surrounding tissue within the anatomical feature of a subject.

In an aspect, a third reservoir is configured and disposed such that it is enabled to apply an initial pressure to a fourth region of the anatomical feature of a subject following deflation of the initial pressure applied to the second region of the anatomical feature of a subject by the first inflatable reservoir and following deflation of the initial pressure applied to the third region of the anatomical feature of a subject by the second inflatable reservoir, enabling thereby detection by the stretchable strain sensing layer of at least one mass having a stiffness different from surrounding tissue within the anatomical feature of a subject.

In an aspect, the tactile sensing system is configured and disposed to enable multiple inflatable reservoirs to be inflated and deflated sequentially in a pattern imitating manual palpation of a breast, wherein the breast is the anatomical feature of a subject.

In an aspect, the tactile sensing system is configured and disposed to enable increasing the pressure from zero to a maximum value and acquiring measurements at intervals of the pressure.

In an aspect, the stretchable strain sensing layer is a continuous sensor, an array of stretchable strain sensing layers, or a combination of continuous sensors and arrays of stretchable strain sensing layers, enabling thereby the formation of an image indicative of the location of at least one mass in the anatomical feature of a subject.

In an aspect, the tactile sensing system is configured to enable injection of currents and the reading of voltages at selectable portions of a stretchable strain sensing layer, wherein the strain sensing layer is continuous.

In an aspect, the formation of an image is enabled by configuring the tactile sensing system to utilize one of electrical impedance tomography and machine learning.

In an aspect, the machine learning includes utilization of one of electrical data or optical data or acoustical data or combinations thereof.

In an aspect, the anatomical contact structure comprises a cup-shaped structure.

In an aspect, as noted, the cup-shaped structure is one of the cup of a brassiere and a male athletic supporter.

In an aspect, the stretchable sensing layer includes a first stretchable sensing layer and a second stretchable sensing layer that are spaced apart from one another, each configured to be disposed in contact with at least one anatomical feature of a subject; and an inflatable reservoir configured and disposed to enable application of pressure to at least one region of an anatomical feature of a subject, enabling thereby the location of at least one mass in the anatomical feature of a subject.

In an aspect, the tactile sensing system includes a computational system wherein the computational system includes a computing device including a processor and a non-transitory memory storing instructions which, when executed by the processor, cause the computing device to, following inflation of an inflatable reservoir: collect data from a stretchable strain sensing layer; and create an image from the data indicative of the amplitude and location of strains in the stretchable strain sensing layer.

In an aspect, the stretchable strain sensing layer is piezoresistive.

The present disclosure relates also to a computational system for diagnosing an anatomical feature of a subject that includes a computing device including a processor and a non-transitory memory storing instructions which, when executed by the processor, cause the computing device to: prior to or during or following inflation of at least one inflatable reservoir to apply pressure to at least one anatomical feature of a subject which may be in conjunction with an anatomical contact structure, collect data from a tactile sensing system in contact with the anatomical feature; and display an image from the tactile sensing system relating to the data collected from the tactile sensing system.

The present disclosure relates also to a tactile sensing system that includes an inflatable reservoir; a stretchable strain sensing layer configured to be disposed in contact with the inflatable reservoir which may be in conjunction with an anatomical contact structure, enabling thereby: indentation of the inflatable reservoir and localized strain in the stretchable strain sensing layer around an indentation or protrusion of the inflatable reservoir; and a computational system that includes: a computing device including a processor and a non-transitory memory storing instructions which, when executed by the processor, cause the computing device to, following inflation of the inflatable reservoir, and as applicable, in conjunction with the anatomical contact structure collect data from the stretchable strain sensing layer and create an image from the data indicative of the amplitude and location of indentations or protrusions of the stretchable strain sensing layer, wherein strain in the stretchable strain sensing layer is caused by touch by an external object or being.

### BRIEF DESCRIPTION OF THE DRAWINGS

The above-mentioned advantages and other advantages will become more apparent from the following detailed description of the various exemplary embodiments of the present disclosure with reference to the drawings, wherein:

FIG. 1A illustrates an embodiment of the tactile sensing system that includes a stretchable strain sensing layer, an inflatable reservoir, and a circuit for detecting a signal from the stretchable strain sensing layer;

FIG. 1B illustrates the embodiment of FIG. 1A with the inflatable reservoir inflated so that the strain sensing layer is thereby stretched, leading to a change in signal;

FIG. 1C illustrates the embodiment of FIG. 1A with the inflatable reservoir inflated and in contact with a protrusion, causing the stretchable strain sensor to stretch around the protrusion and the reservoir to be indented;

FIG. 1D illustrates an embodiment of the tactile sensing system that includes a stretchable strain sensing layer, a compliant layer, an inflatable reservoir that is less compliant than the strain sensing and compliant layers, and a circuit for detecting a signal from the stretchable strain sensing layer;

FIG. 1E illustrates the embodiment of FIG. 1D with the inflatable reservoir inflated;

FIG. 1F illustrates the embodiment of FIG. 1D with the inflatable reservoir inflated and in contact with a protrusion, causing the stretchable strain sensor to stretch around the protrusion and the compliant layer to be indented;

FIG. 1G illustrates an embodiment of the tactile sensing system that includes a stretchable strain sensing layer, an inflatable reservoir, and a system capable of creating images showing the amplitudes and spatial locations of strain in the strain sensing layer, the lack of strain being shown as a featureless image;

FIG. 1H illustrates the embodiment of FIG. 1G with the inflatable reservoir inflated and in contact with a protrusion, causing the stretchable strain sensor to stretch around the protrusion and the compliant layer to be indented, the location and amplitude of the strain around the protrusion being shown in an image;

FIG. 1I illustrates an embodiment of the tactile sensing system that includes a stretchable strain sensing layer, an inflated reservoir where the reservoir is the arm of an inflatable robot, and a system capable of creating images showing the amplitudes and spatial locations of strain in the strain sensing layer, the lack of strain being shown as a featureless image;

FIG. 1J illustrates the embodiment of FIG. 1I with the inflatable arm reservoir inflated and in contact with a protrusion, causing the stretchable strain sensor to stretch around the protrusion and the compliant layer to be indented, the location and amplitude of the strain around the protrusion being shown in an image;

FIG. 2A illustrates the tactile sensing system in which the stretchable sensing layer and the inflatable reservoir are separate components;

FIG. 2B illustrates the embodiment of FIG. 2A with the inflatable reservoir in contact with the stretchable strain sensing layer under inflation;

FIG. 2C illustrates the deformation of the inflatable reservoir in the embodiment of FIG. 2A under inflation in directions in which it is not mechanically restrained;

FIG. 2D illustrates the tactile sensing system in which the stretchable sensing layer and the inflatable reservoir are integrated as a multi-component structural member;

FIG. 2D′ is an enlarged view of Detail 2D′ in FIG. 2D showing the strain sensing layer positioned on the interior surface of a wall of the inflatable reservoir;

FIG. 2D″ is an enlarged view of Detail 2D″ in FIG. 2D showing the strain sensing layer positioned on the exterior surface of a wall of the reservoir;

FIG. 2E illustrates an embodiment of the tactile sensing system in which the strain sensing layer is disposed on a reservoir with a protruding shape that could be inserted into a cavity;

FIG. 2F illustrates the embodiment of FIG. 2E in the inflated state;

FIG. 3A illustrates a stretchable sensing layer in the form of a continuous area;

FIG. 3B illustrates a stretchable sensing layer in the form of a strip;

FIG. 3C illustrates a stretchable sensing layer in the form of a serpentine;

FIG. 3D illustrates a stretchable sensing layer in the form of an array of strip-shaped areas;

FIG. 3E illustrates a stretchable sensing layer in the form of an array of strip-shaped areas in rows and columns;

FIG. 3F illustrates a stretchable sensing layer in the form of an array of individual elements;

FIG. 4A illustrates an embodiment of the tactile sensing system comprising a strain sensing layer, an inflatable reservoir, and a non-stretchable cup-shaped structure;

FIG. 4B illustrates an embodiment of the tactile sensing system in which the stretchable strain sensing layer and the non-stretchable cup-shaped structure are joined to form an inflatable reservoir;

FIG. 4C illustrates how the reservoir expands in the direction of the stretchable strain sensing layer but not in the direction of the non-stretchable cup-shaped structure under inflation;

FIG. 4D illustrates an embodiment of the tactile sensing system configured as a tumor detection system that includes an anatomical contact structure configured to allow the strain sensing layer to be in contact with at least one part of the body of the patient and configured to allow the inflatable reservoir to apply pressure to at least one part of the body;

FIG. 4E illustrates a cross-sectional close-up of the tumor detection embodiment of FIG. 4D placed on a breast in the non-inflated state;

FIG. 4F illustrates a cross-sectional close-up of the tumor detection embodiment of FIG. 4D deforming the breast in the inflated state;

FIG. 5A illustrates a tissue of uniform stiffness under compression by the inflatable reservoir in FIG. 4F;

FIG. 5B illustrates a tissue containing a hard mass or “lump” experiencing a deformation due to the inflation of the inflatable reservoir in FIG. 4F that leads to a strain in the strain-sensing layer;

FIG. 5C illustrates a tumor detection system having two stretchable sensing layers and two inflatable reservoirs in the uninflated state on a tissue of uniform stiffness;

FIG. 5D illustrates the tumor detection system from FIG. 5C with one of the two inflatable reservoirs in the inflated state compressing a tissue of uniform stiffness;

FIG. 5E illustrates the tumor detection system from FIG. 5C with one of the two inflatable reservoirs in the inflated state compressing a tissue with a hard mass, leading to a difference in the strains in the sensing layers compared to FIG. 5D;

FIG. 5F shows a cross-sectional illustration of a tumor detection system having one stretchable sensing layer and multiple inflatable reservoirs in the uninflated state;

FIG. 5G shows a frontal view of the multiple inflatable reservoirs and the strain sensing layer of FIG. 5F;

FIG. 5H shows a cross-sectional illustration of a tumor detection system having one stretchable sensing layer and multiple inflatable reservoir wherein the area of the stretchable sensing layer is significantly less than the sum of the areas of the multiple inflatable reservoirs.

FIG. 5I shows a frontal view of the embodiment of FIG. 5H in which the strain sensing layer underlies only some of the reservoirs or only parts of some of the reservoirs;

FIG. 6A is a 3-dimensional partially transparent view of an embodiment of the tactile sensing system configured as a tumor detection system as shown in FIGS. 4A-4F for the purpose of benchtop testing with a phantom;

FIG. 6B is a cross-sectional view of the embodiment shown in FIG. 6A;

FIG. 6C shows a continuous stretchable strain sensor disposed over a lifeform that is a phantom breast; the sensor being in electrical communication with a circuit via electrical leads attached at 16 points around its periphery;

FIG. 6D shows a bottom view of a breast phantom with phantom tumor masses;

FIG. 7A illustrates EIT images showing locations of changes in conductivity of the stretchable strain sensor as a result of localized stretching as a consequence of the application of pressure to a breast phantom as illustrated in FIGS. 6A and 6B;

FIG. 7B shows a close-up of the image taken at 80 mm Hg with the phantom containing two (2) lumps;

FIG. 7C shows a contour plot of the data in FIG. 7B, taken from a phantom with two lumps at 80 mm Hg of air pressure;

FIG. 8A illustrates an array of strip-shaped sensors formed from a series of orthogonally positioned crossing strips of eight (8) rows and eight (8) columns as illustrated in FIG. 3E;

FIG. 8B illustrates the corresponding image produced in response to a stretch imposed at row 4, column 4;

FIG. 9 illustrates an embodiment of hardware and software used to collect data and display an image from the tactile sensing system;

FIG. 10A illustrates an embodiment of strip-shaped sensors in the un-stretched and stretched states;

FIG. 10B illustrates the change in resistance as a function of strain for a strip-shaped sensor;

FIG. 11A illustrates a distributed sensing system wherein electrical impedance tomography (EIT) is utilized to image a continuous sensor area wherein the hardware to collect EIT data includes multiplexers and a controller to change the points at which currents are input and voltages are measured.

FIG. 11B illustrates the process for obtaining the strain distribution from the voltages measured using the hardware of FIG. 11A;

FIG. 12A illustrates the starting material of acid-intercalated graphite in a method of manufacturing a piezoelectric sensing layer comprising exfoliated graphite (EG) and latex (latex/EG);

FIG. 12B illustrates the exfoliated graphite produced in a method of manufacturing a piezoelectric latex/EG sensing layer;

FIG. 12C illustrates the sprayable solution produced in a method of manufacturing a piezoelectric latex/EG sensing layer;

FIG. 12D illustrates the spraying of the solution of FIG. 12C in a method of manufacturing a piezoelectric latex/EG sensing layer;

FIG. 12E illustrates the a large-area piezoelectric latex/EG sensing layer coated onto a latex membrane in a method of manufacturing a sensing layer; and

FIG. 12F illustrates a method of forming an electrical connection to a piezoelectric latex/EG sensing layer.

### DETAILED DESCRIPTION

For the purposes of promoting an understanding of the principles of the present disclosure, reference will now be made to the exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The word “exemplary” is used herein to mean “serving as an example, instance, or illustration.” Any embodiment described herein as “exemplary” is not necessarily to be construed as preferred or advantageous over other embodiments.

It is to be understood that the method steps described herein need not necessarily be performed in the order as described. Further, words such as “thereafter,” “then,” “next,” etc., are not intended to limit the order of the steps. Such words are simply used to guide the reader through the description of the method steps.

The implementations described herein may be implemented in, for example, a method or a process, an apparatus, a software program, a data stream, or a signal. Even if only discussed in the context of a single form of implementation (for example, discussed only as a method), the implementation of features discussed may also be implemented in other forms (for example, an apparatus or program). An apparatus may be implemented in, or in conjunction with, for example, appropriate hardware, software, or firmware, or a combination or sub-combination thereof. The methods may be implemented in, for example, an apparatus such as, for example, a processor, which refers to processing devices in general, including, for example, a computer, a microprocessor, an integrated circuit, or a programmable logic device. Processors also include communication devices, such as, for example, computers, cell phones, tablets, portable/personal digital assistants, and other devices that facilitate communication of information between end-users within a network.

The general features and aspects of the present disclosure remain generally consistent regardless of the particular purpose. Further, the features and aspects of the present disclosure may be implemented in a system in any suitable fashion, e.g., via the hardware and software configuration of system or using any other suitable software, firmware, and/or hardware. For instance, when implemented via executable instructions, such as the set of instructions, various elements of the present disclosure are in essence the code defining the operations of such various elements. The executable instructions or code may be obtained from a computer-readable medium (e.g., a hard drive media, optical media, EPROM, EEPROM, tape media, cartridge media, flash memory, ROM, memory stick, and/or the like) or communicated via a data signal from a communication medium (e.g., the Internet). In fact, readable media may include any medium that may store or transfer information.

A tactile sensing system, according to the present disclosure, includes a stretchable strain sensing layer. In one embodiment, the stretchable strain sensing layer comprises a stretchable piezoresistive material. Piezoresistors change electrical resistivity when they are stretched. As is known in the art, piezoresistivity refers to a change in the electrical resistivity of a material under strain, to be distinguished from changes in resistance due to dimensional changes of a resistor that is strained.

In an embodiment, the electrical conductivity of the piezoresistive material is due to percolation pathways formed between conductive filler particles in an insulating host matrix. In an embodiment, the piezoresistivity is due to changes in the positions of the filler particles during strain, which leads to changes in the percolation pathways. In an embodiment, the piezoresistor is formed from a stretchable insulating polymer composite containing conductive or semiconductive filler particles. In an embodiment, the conductive or semiconductive filler particles are nano-carbon. In one embodiment, a composite of exfoliated graphite (EG) mixed into latex is used as the stretchable piezoresistive material.

In one embodiment, the stretchable piezoresistive material is applied as a thin film onto a stretchable substrate. Examples of stretchable substrates include latex sheets.

In an embodiment, the stretchable strain sensing layer comprises a fabric coated with an electrically conductive or semiconductive material. Fabrics coated with conductive or semiconductive materials change their electrical resistance when they are stretched due to changes in the strength of electrical contacts between fibers in the fabric. Electrical leads are attached to the stretchable piezoresistive strain sensing layer to allow its resistance to be monitored.

Other types of strain sensing layers may be used, as known to those persons having ordinary skill in the art. In an embodiment, the strain sensing layer may be based on electrical properties such as capacitance or piezoelectric properties. In an embodiment the strain sensing layer may be based on optical properties. In an embodiment, the strain sensing layer may comprise optical fibers, which change their light-carrying ability when bent. Optical fibers, as well as fibers or films made from other non-stretchable materials, may be made stretchable by disposing them in serpentine shapes.

A tactile sensing system, according to the present disclosure, includes an inflatable reservoir or air bladder. The reservoir may be shaped appropriately for the surface to be sensed. For example, it may have a concave surface to make contact with a a protruding feature, a flat surface for making contact with substantially flat object, or a bulb shape to fit into a cavity.

In an embodiment, the present disclosure relates to an automated device for palpation for the detection of tumors that are stiffer than the surrounding tissue. The device comprises both hardware and software and includes a continuous sensing area. The stretchable sensing layer conforms to the tissue or organ. This automated palpation system mimics a clinical exam, without requiring a healthcare professional trained in palpation.

In an embodiment, the present disclosure relates to an automated device for breast palpation for the detection of breast tumors that are stiffer than the surrounding tissue. The embodiment includes one or more piezoresistive sensing layers and one or more inflatable reservoirs built into a brassiere, along with a portable electronic system.

The piezoresistive material according to the present disclosure is a conductive composite comprising conductive carbon nanoparticles embedded in latex, the latter serving as an insulating host material. The amount of conductive carbon in the composite is high enough that an electrical pathway is formed through the insulating host. When this material is stretched, some of the conducting pathways are broken as a result of a separation of some of the carbon particles, causing the resistance to increase. The material is painted onto a rubber sheet by spray-coating to form a free-standing stretchable strain sensing layer.

Sensing over an area may be accomplished by using serpentines, arrays, or continuous sensing areas. Serpentines are typical in commercial thin film metal strain gauges. When using continuous sensing areas, the electrical resistance in the interior of the area may be determined using various data collection methods and algorithms.

Electrical impedance tomography is an imaging technique in which currents are injected at various locations at the periphery of an electrically resistive area, voltages are measured at various locations on the periphery, and an algorithm is used to determine changes in the conductivity from a baseline state from those measurements.

An alternative disposition of the sensing material is in the form of an array of multiple discrete elements, rather than a continuous area. The elements may be in the form of strips that make up rows, or rows and columns. The elements may be in the form of discrete elements covering a small area of the surface.

To detect stiffness difference on the soft tissue of, for example, a breast, a pressurization system is required to press the strain sensing material against the breast to cause the stretchable strain sensing layer to deform due to the presence of the malignant tissue.

As defined herein, an anatomical feature of a subject includes tissue or other parts of the anatomy of a human being or a pet or animal. It may also be considered to include a part of an inanimate object, such as for example a robot or robotic mechanism. The tissue may be located in a region of the anatomical feature of a subject, for example, in a region of the breast or testicle or in a region of an arm, leg, neck, chest, etc.

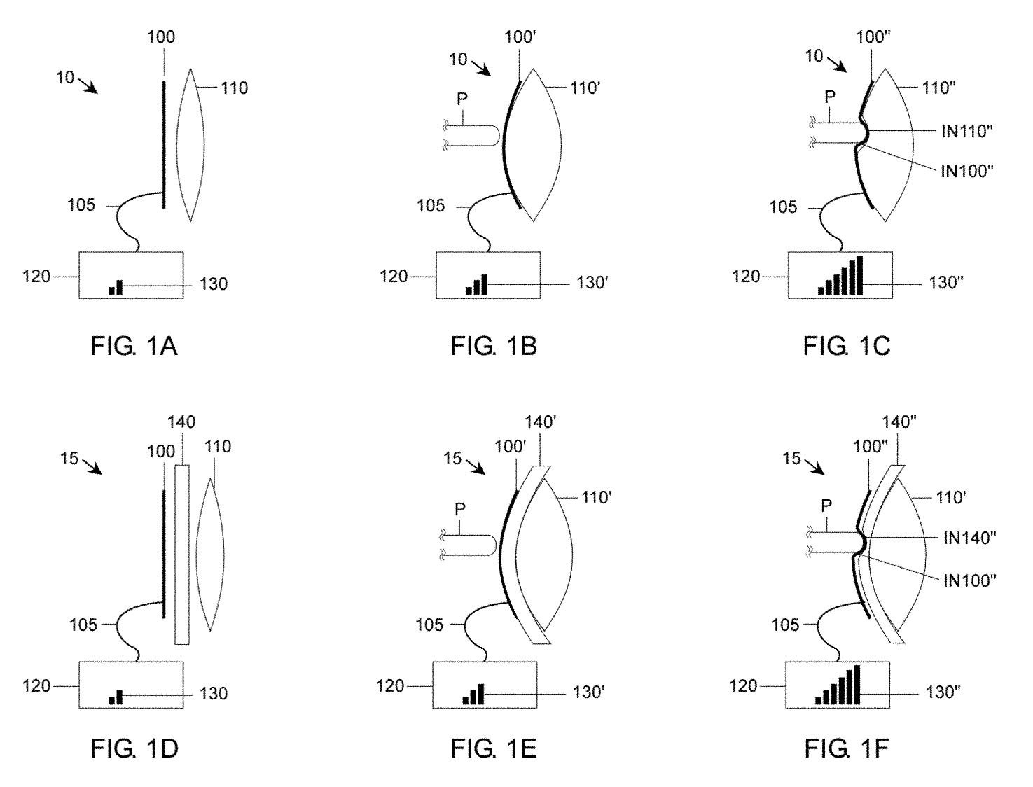

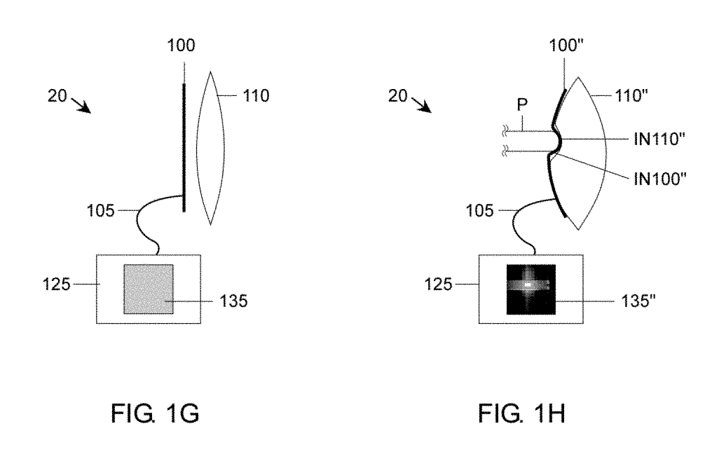

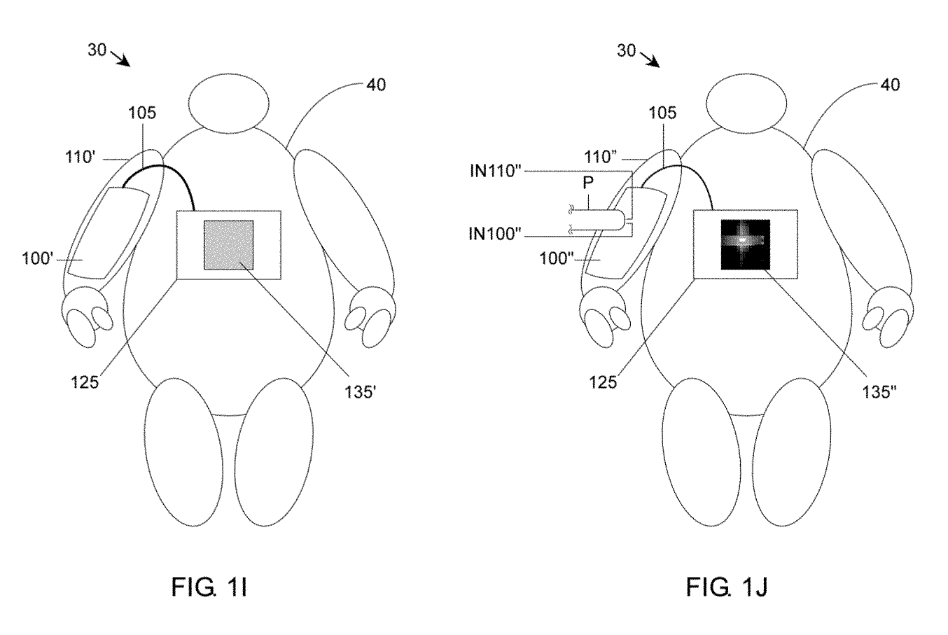

FIG. 1A illustrates an embodiment of a tactile sensing system 10 in cross-section. It includes a stretchable strain sensing layer 100, an inflatable reservoir 110 having a convex profile, and a circuit 120 for detecting a signal 130 from the strain sensing layer via connection 105.

FIG. 1B illustrates the tactile system 10 of FIG. 1A with reservoir 110′ in the inflated state. Strain sensing layer 100′ has been slightly stretched by the inflation, causing a small change in the signal from 130 to 130′.

FIG. 1C illustrates the tactile system 10 of FIG. 1A with reservoir 110″ in the inflated state and indented as indentation IN110″ due to contact with a protrusion P from an object (the rest of which is not shown). Strain sensing layer 100″ has been stretched locally as indentation IN100″ around the protrusion P, causing a change in the signal 130″.

FIG. 1D illustrates an embodiment of a tactile sensing system 15 in which inflatable reservoir 110 is not readily indented and a compliant layer 140 that is readily indented is disposed under the strain sensing layer.

FIG. 1E illustrates the tactile system 15 of FIG. 1D with reservoir 110′ in the inflated state.

FIG. 1F illustrates the tactile system 15 of FIG. 1D with reservoir 110′ in the inflated state and compliant layer 140″ indented as indentation IN140″ due to contact with a protrusion P from an object. Strain sensing layer 100″ has been stretched locally as indentation IN100″ around protrusion P, causing a change in the signal 130″.

FIG. 1G illustrates a tactile system 20 that includes a system 125 capable of creating an image 135 showing the amplitudes and spatial locations of strain in strain sensing layer 100.

FIG. 1H illustrates the tactile system 20 in which strain sensing layer 100″ has been stretched locally as indentation IN100″ at indentation IN110″ in reservoir 110″ by protrusion P, and image 135″ shows the location and amplitude of the strain.

FIG. 1I illustrates a tactile sensing system 30 wherein strain sensing layer 100′ is disposed on an arm 110′ of a robot 40 and wherein robot 40 and arm 110′ are inflated. Strain sensing layer 100′ is in communication with an image-creating system 125.

FIG. 1J illustrates the robot tactile system 30 wherein strain sensing layer 100″ been stretched locally as indentation IN100″ at indentation IN110″ in reservoir 110″ by protrusion P, as indicated by image 135″.

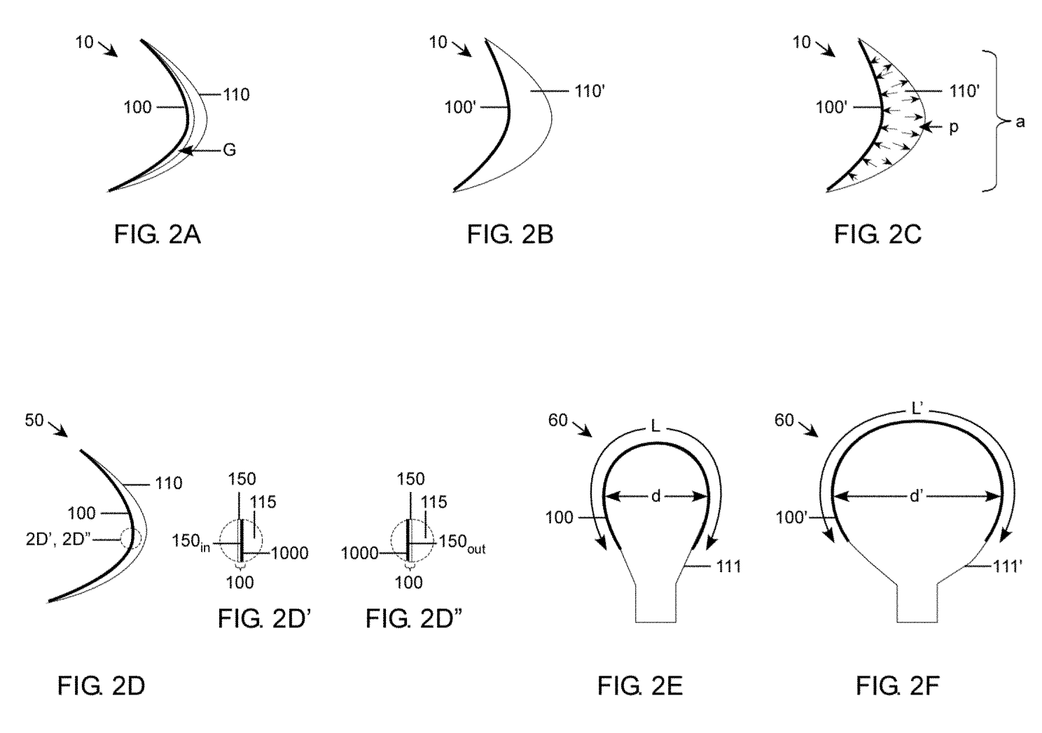

FIG. 2A illustrates an aspect of the tactile sensing system 10 of FIG. 1A in which stretchable strain sensing layer 100 is a separate component from inflatable reservoir 110 such that a gap G is formed between layer 100 and reservoir 110.

FIG. 2B illustrates the aspect of FIG. 2A in which reservoir 110′ is in the inflated state and in contact with stretchable strain sensing layer 100′ causing gap G in FIG. 2A to disappear.

FIG. 2C illustrates that inflated reservoir 110′ of FIG. 2B has expanded in all directions, as indicated by arrows a, under pressure p.

FIG. 2D illustrates an aspect of the tactile sensing system 10 in which stretchable strain sensing layer 100 is integrated with inflatable reservoir 110, for example forming one wall 150 of reservoir 110, forming thereby a multi-component structural member 50. The integration may be achieved by applying a coating 1000 to wall 150.

FIGS. 2D′ and 2D″ are detailed views which show that a strain sensing coating 1000 may be positioned either in the interior space 115 of reservoir 110 by placement on an interior surface 150ᵢₙ of wall 150 of reservoir 110 as shown in FIG. 2D′, or on the outside of reservoir 110 by placement on an exterior surface 150ₒᵤₜ of wall 150 as shown in FIG. 2D″. In this embodiment coating 1000 and wall 150 together comprise strain sensing layer 100.

FIG. 2E illustrates an aspect 60 of the tactile sensing system 10 of FIG. 1A in which strain sensing layer 100 is disposed on a protruding bulb-shaped reservoir 111 having a cross-sectional dimension d in a non-inflated condition wherein strain sensing layer 100 has an unstressed cross-sectional length L.

FIG. 2F illustrates the change in shape of the aspect 60 of FIG. 2E after expansion of bulb-shaped reservoir 111 to an expanded condition 111′ due to inflation, now having a cross-sectional dimension d′ in an inflated condition wherein strain sensing layer 100 in the expanded condition 100′ has a stressed cross-sectional length L′ due to the increase in volume of reservoir 111′.

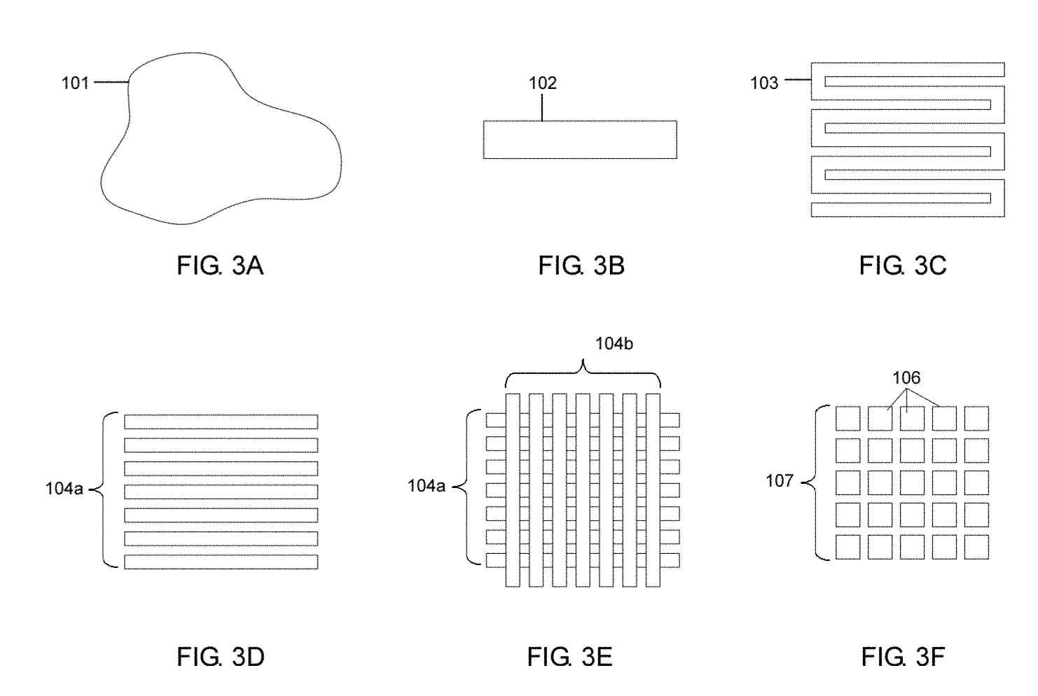

FIG. 3A illustrates a stretchable sensing layer 100 in the form of a continuous area 101. Typical shapes for continuous sensors are square, rectangular, and circular, but other shapes can be used.

FIG. 3B illustrates a stretchable sensing layer 100 in the form of a rectangular strip 102.

FIG. 3C illustrates a stretchable sensing layer 100 in the form of a serpentine 103.

FIG. 3D illustrates a stretchable sensing layer 100 in the form of an array of strip-shaped areas 104a disposed in rows.

FIG. 3E illustrates a stretchable sensing layer in the form of an array of strip-shaped areas disposed in rows 104a and columns 104b. The rows and columns may be in the form of patterned coatings on opposite sides of a stretchable support or membrane, or the rows may be disposed on a first stretchable support and the columns may be disposed on a second stretchable support, or the rows and columns may be formed from individual strip-shaped sensing layers, or the rows and columns may be formed on one stretchable support with an intervening layer between them of a material that prevents signal communication between the rows and columns.

FIG. 3F illustrates a stretchable sensing layer 100 in the form of an array 107 of individual elements 106.

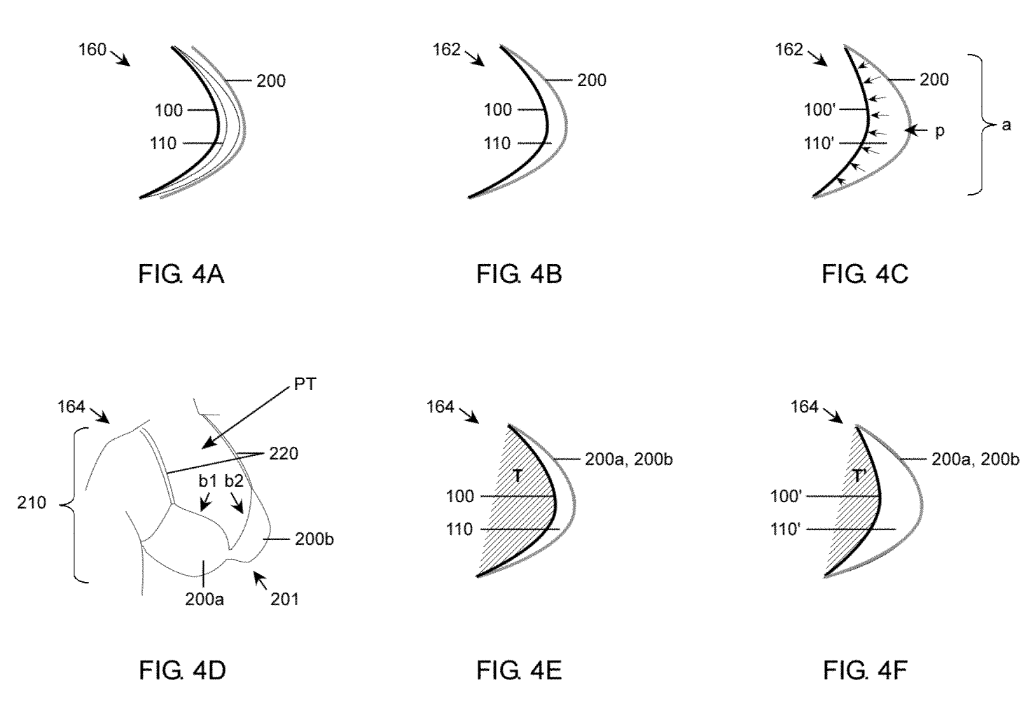

FIG. 4A illustrates an aspect 160 of tactile sensing system 10 further comprising a substantially non-stretchable structure in the form of a cup 200. In this embodiment, sensing layer 100, reservoir 110, and cup-shaped structure 200 are separate components wherein reservoir 110 is positioned between sensing layer 100 and cup-shaped structure 200.

FIG. 4B illustrates an aspect 162 of the tactile sensing system 10 in which sensing layer 100, reservoir 110, and cup-shaped structure 200 are integrated as a unitary system whose components move in unison. For example, sensing layer 100 and cup-shaped structure 200 may be walls defining an interior space to create reservoir 110.

FIG. 4C illustrates how non-stretchable structure 200 of FIG. 4B restricts expansion due to pressure p, indicated by arrows a, of reservoir 110′ defined by sensing layer 100′ and cup 200.

FIG. 4D illustrates a tactile sensing system 164 that is configured as a tumor detection system for detecting breast cancer. System 164 includes at least one stretchable strain sensing layer 100 (see FIGS. 4A-4C) in contact with one breast b1 or b2 or both breasts b1 and b2 of a patient PT. System 164 further includes an anatomical contact structure 210, here a brassiere 201 with breast cups 200a and 200b, each containing at least one inflatable reservoir 110, and straps with fasteners 220. Contact structure 210 is configured to allow inflatable reservoir 110 (see FIGS. 4A-4C) to apply pressure to the breast tissue T, as shown in FIGS. 4E-4F, through the attachment of cups 200a and 200b to the body of patient PT using straps 220 and other parts of brassiere 201. An inflatable reservoir 110 can be positioned over the breast b1 or b2 or both breasts b1 and b2 in the non-inflated configuration 110 and then inflated to configuration 110′, causing breast tissue T′ to deform. Thus, anatomical contact structure 210 is configured to enable inflatable reservoir 110 to apply pressure to a region of the anatomical feature of the subject. Thus inflatable reservoir 110 is enabled to apply pressure to a region of the anatomical feature of the subject in conjunction with anatomical contact structure 210.

Consequently, tactile sensing system 164 is configured as a tumor detection system that includes anatomical contact structure 210 configured to allow strain sensing layer 100 to be in contact with at least one part of the body of patient PT and configured to allow inflatable reservoir 110 to apply pressure to at least one part of the body, which may be considered to be an anatomical feature of a subject. As an example, the part of the body is one or both breasts b1 and b2 and the anatomical contact structure is the bra 201. Inflatable reservoir 110 is formed by stretchable tactile sensing layer 100 and bra cup 200a or 200b.

FIG. 4E illustrates a close-up view of tactile sensing system 164 showing the positioning of bra cup 200a or 200b with sensing layer 100 and inflatable reservoir 110 over breast b1 or b2 in a non-inflated configuration 110, where tissue T is not compressed.

FIG. 4F illustrates the deformation of breast tissue T′ under pressure from inflatable reservoir 110′ of the palpation brassiere tumor detecting system 164 of FIG. 4D.

As defined herein, an anatomical contact structure includes garments such as bras. It may also include structures configured to facilitate manual placement of the tactile system. It may also include automated systems for positioning the tactile system, such as the robot of FIG. 1J: the tactile system on the robot arm may be placed into an appropriate position to allow a strain sensor to be in contact with a region of an anatomical feature of a subject and for an inflatable reservoir to apply pressure to an anatomical feature of a subject by the motion of the robot. In this aspect, the robot functions as an anatomical contact structure.

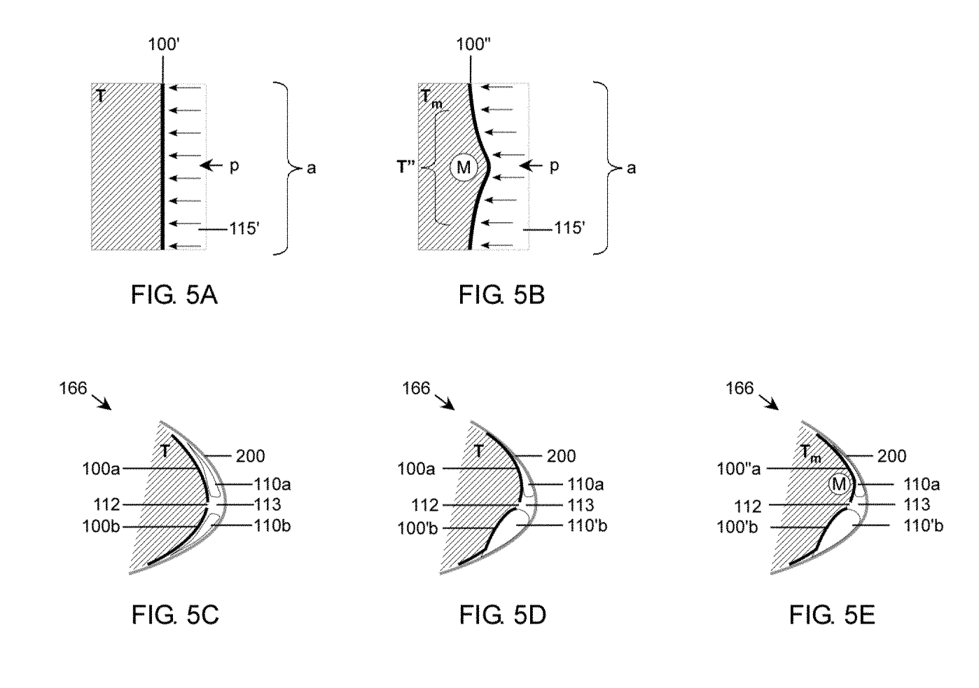

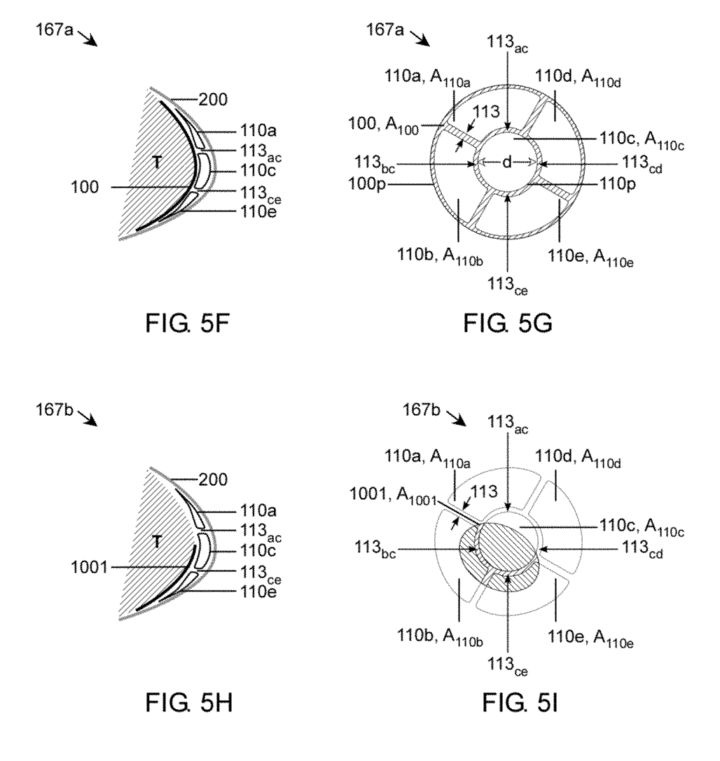

FIG. 5A shows the interior space of an inflated reservoir 115′ applying a pressure p to a tissue T of uniform stiffness. The expansion of interior space 115′ of the reservoir is indicated by arrows a. There is little strain in stretchable sensing layer 100′.

FIG. 5B shows interior space 115′ applying pressure p to a tissue Tₘ containing a hard mass M. Stretchable strain sensing layer 100″ is locally strained around a tissue deformation area T″ when pressure from the inflatable reservoir is applied (see also FIG. 4F). In the absence of a hard mass, as shown in FIG. 5A, there is no corresponding localized strain in stretchable strain sensing layer 100′.

It should be appreciated that while FIG. 5B shows a local strain around a mass with a stiffness greater than the surrounding tissue, strain may also occur around a mass with a stiffness lower than the surrounding tissue if the reservoir protrudes into the area of softer tissue leading to a local strain in the strain sensing layer. A mass with a stiffness lower than the surrounding tissue may include a void.

FIG. 5C illustrates a tactile sensing system 166 configured as a tumor detection system having two stretchable sensing layers 100a and 100b separated by a gap 112 and two inflatable reservoirs 110a and 110b in the uninflated state separated by a gap 113 disposed within bra cup 200. Additional sensing layers and reservoirs may be used. In the illustration, two inflatable reservoirs 110a and 110b are shown, but multiple reservoirs may be employed. In the illustration, stretchable strain sensing layers 100a and 100b are continuous (see FIG. 3A), but alternative configurations can be used, such as a matrix of strip-shaped strain sensing layers such as shown in FIGS. 3B-3E. In the illustration of FIG. 5C, stretchable strain sensing layers 110a and 110b are shown as adjacent, but they may be overlapping. Stretchable strain sensing layers 100a and 100b are configured and disposed to enable contact with a region of an anatomical feature of a subject, e.g., tissue T in FIG. 5C.

Thus, a first inflatable reservoir 110a is configured and disposed to apply pressure to part of an anatomical feature of a subject, e.g., tissue T of patient PT, and a second inflatable reservoir 110b is configured and disposed to apply pressure to a different part of the same anatomical feature of a subject, e.g., tissue T of patient PT.

First inflatable reservoir 110a is configured and disposed with respect to second inflatable reservoir 110b to enable differential application of pressure to different parts of the anatomical feature of the subject, e.g., tissue T, thereby increasing the probability of detection by the tactile sensing system 166 of a tumor within the anatomical feature of the subject.

Gaps 112 and 113 may have a zero distance dimension such that adjacent stretchable strain sensing layers 100a and 100b may be in contact with one another or entirely contiguous or overlapping.

FIG. 5D illustrates the tactile sensing system 166 configured as a tumor detection system from FIG. 5C with one of the two inflatable reservoirs, 110′b, in the inflated state. FIG. 5D illustrates only reservoir 110′b in the inflated state, but multiple reservoirs may be simultaneously inflated. The use of multiple reservoirs mimics the motion of the hand over the tissue during palpation if the reservoirs are inflated in different combinations, such as none inflated, 110′a only inflated, 110′b only inflated, 110′a and 110′b both inflated, etc.

FIG. 5E illustrates the tumor detection system from FIG. 5C with one of the two inflatable reservoirs, 110′b, in the inflated state, leading to a deformation of the tissue Tₘ and strains in one or more of sensing layers 100″a or 100′b. In FIG. 5E, strain sensing layer 100″a is locally strained around mass M, but strain sensing layer 100′b is not. Different inflation patterns will lead to different signals coming from the strain sensing layers 100, permitting better detection of mass M.

FIG. 5F illustrates a cross-sectional view of a tactile sensing system 167a configured as a tumor detection system having multiple reservoirs 110a, 110c, and 110e and a single strain sensing layer 100. Reservoirs 110a and 110c are separated by gap 113ac while reservoirs 110c and 110e are separated by gap 113ce.

FIG. 5G shows a frontal view of tactile sensing system 167a having multiple arc-shaped reservoirs 110a-110b and 110d-110e positioned around a central circular reservoir 110c to follow the contour of breast b1 or b2 in FIG. 5F. Gaps 113 occur tangentially between arc-shaped reservoirs 110a-110b and 110d-110e positioned around central circular reservoir 110c, while gaps 113ac, 113bc, 113cd, and 113ce occur radially between central circular reservoir 110c and arc-shaped reservoirs 110a-110b and 110d-110e, respectively. While reservoirs 110a-110b and 110d-110e are illustrated as arc-shaped and central reservoir 110c is illustrated as circular, other shapes such as triangular, elliptical, polygonal, or other suitable shapes may be utilized.

An area of correspondence between a single reservoir and a single strain sensor may be defined as the area A₁₁₀ of the surface of reservoirs 110 divided by the area A₁₀₀ of strain sensing layer 100 with which reservoir 110 is in contact, i.e. A₁₁₀/A₁₀₀, if A₁₁₀ is less than A₁₀₀. If A₁₁₀ is greater than A₁₀₀, then the area of correspondence is alternatively defined as the inverse of that, A₁₀₀/A₁₁₀.

For the case of a circular central reservoir such as reservoir 110c in FIG. 5G, because the outer peripheral edge 100p of strain sensing layer 100 extends beyond the outer peripheral edges 110p of reservoir 110c, the area of correspondence is equal to A_{110c}/A₁₀₀, where A_{110c} is the area of reservoir 110c, determined by its diameter d, and A₁₀₀ is the area of the sensor. The area of correspondence is less than 100% because the reservoir is smaller than the sensor.

An area of correspondence between a first set of reservoirs and a second set of strain sensors may likewise be defined as the sum of the areas A₁₁₀ of the surfaces of all the reservoirs in the first set and the sum of the areas A₁₀₀ of all the strain sensing layers in the second set. For the case of the five reservoirs 110a-e and the one strain sensor 100 in FIG. 5G, the area of correspondence is equal to the sum A₁₁₀ₐ+A_{110b}+A_{110c}+A_{110d}+A₁₁₀ₑ divided by A₁₀₀.

Therefore, in FIG. 5G, although opposing surfaces of each of the reservoirs 110a-110e are in contact with the surface of strain sensing layer 100, the area of correspondence is less than 100% due to the presence of gaps 113 and 113ac-113ce and the portion of strain sensing layer 100 which extends beyond the outer edges of arc-shaped reservoirs 110a-110b and 110d-110e.

Due to gaps 113 and 113ac-113ce, a portion of strain sensing layer 1001 is initially exposed. Gaps 113 and 113ac-113ce may be varied depending on the amount of inflation of reservoirs 110a-110e, and the gaps may be zero either initially or during the diagnostic evaluation provided by tactile sensing system 167a.

FIG. 5H illustrates a cross-sectional view of a tactile sensing system 167b configured as a tumor detection system having multiple reservoirs 110a, 110b, and 110c and a single strain sensing layer 1001. Reservoirs 110a and 110c are separated by gap 113ac while reservoirs 110c and 110e are separated by gap 113ce.

FIG. 5I shows a frontal view of tactile sensing system 167b wherein strain sensing layer 1001 has an oval or elliptical shape and an area A₁₀₀₁ that is smaller than the sum of areas A₁₁₀ₐ-A₁₁₀ₑ. Only portions of reservoirs 110b, 110c and 110e are positioned over strain sensing layer 1001.

Thus, the multiple reservoirs 110a-e are disposed with strain sensing layer 1001 disposed partially under reservoirs 110b, 110c, and 110e. As can be understood, the number, size, and placement of the reservoirs and the strain sensing layers may differ.

Consequently, at least a portion of the region of the anatomical feature to which pressure is enabled to be applied by inflatable reservoirs 110a to 110e at least partially corresponds to the portion of the region of the anatomical feature with which stretchable strain sensing layer 100 or 1001 is in contact.

In an aspect of the present disclosure, the region of the anatomical feature to which pressure is enabled to be applied by one or more inflatable reservoirs 110a to 110e does not correspond to the region of the anatomical feature with which one or more stretchable strain sensing layers 100 or 1001 are in contact. In this aspect, strain sensing layer 1001 would be disposed outside of the region defined by inflatable reservoirs 110a to 110e in FIG. 5I.

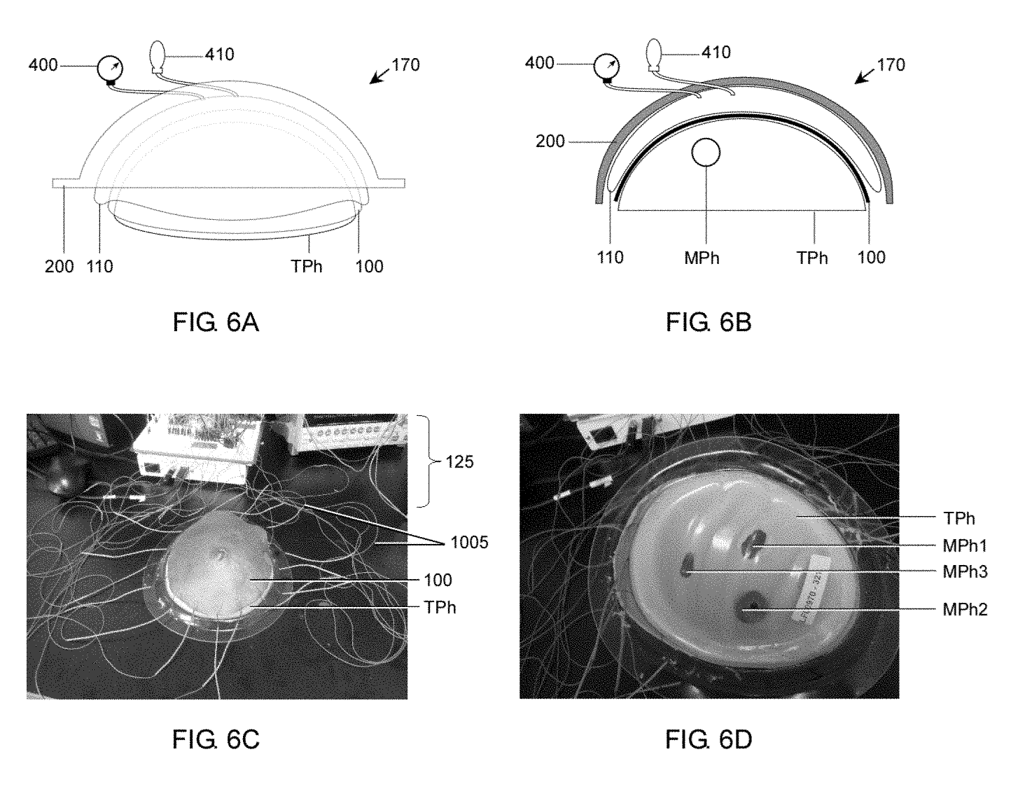

FIG. 6A shows a 3-dimensional partially transparent view of an embodiment of a tumor detection system for benchtop testing with a lifeform or tissue phantom TPh. Stretchable strain sensing layer 100 is conformal to, and disposed over, phantom TP. Sensing layer 100 is surmounted by balloon or inflatable reservoir 110 having a pressurization bulb 410 and a pressure gauge or manometer 400. Balloon 110 is surmounted by cup-shaped support structure 200 in the form of a rigid bowl-like structure.

FIG. 6B is a cross-sectional view of the embodiment shown in FIG. 6A. The tissue phantom TPh contains a phantom mass MPh.

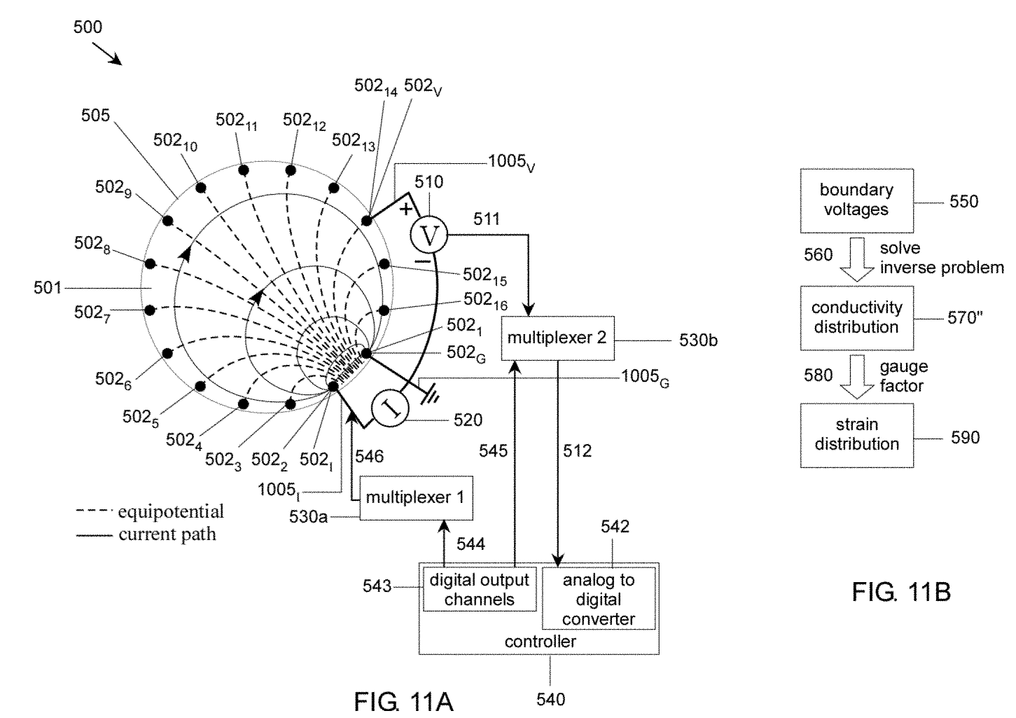

FIG. 6C shows an actual photograph of continuous stretchable strain sensor 100 disposed over a lifeform that is phantom breast TPh. Strain sensing layer 100 is formed from a piezoresistive layer coated onto a rubber membrane. Stretching strain sensing layer 100 results in changes in its resistance R, or alternatively its conductance S, where S=1/R. Strain sensing layer 100 is in electrical communication with an imaging system 125 via electrical leads 1005 attached at 16 points around its perimeter, e.g., at 16 points of electrodes 502₁ to 502₁₆ around perimeter 505 of the strain sensing layer 501 as described below with respect to FIGS. 11A-11B.

FIG. 6D shows a bottom view of breast phantom TPh with phantom tumor masses MP1, MP2 and MP3.

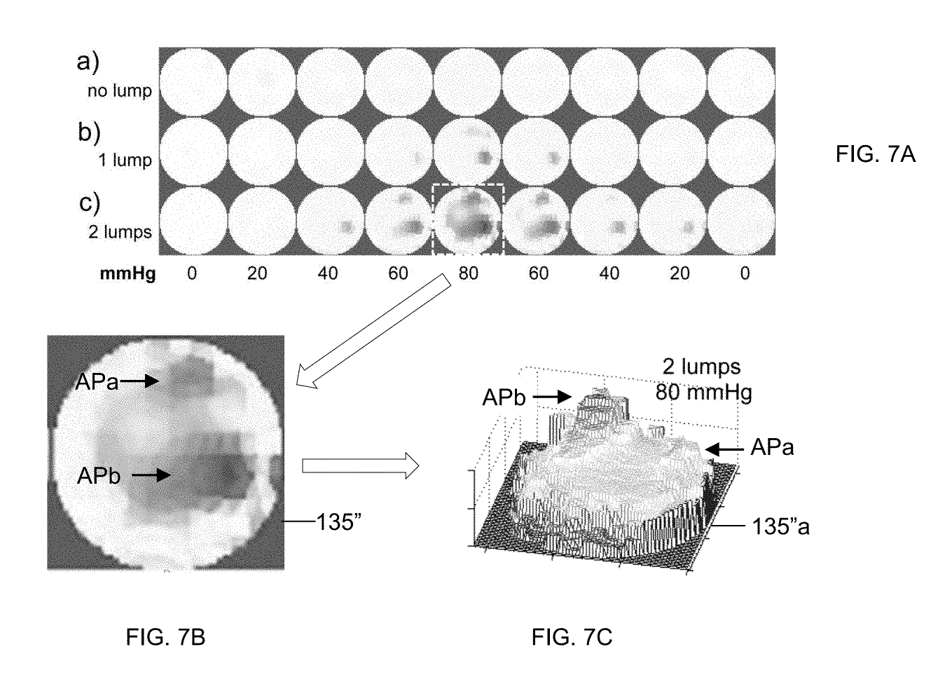

FIG. 7A illustrates electrical impedance tomography (EIT) images showing locations of increases in resistance (red) over the area of a continuous stretchable strain sensor as a result of localized stretching as a consequence of the application of pressure to a breast phantom as illustrated in FIGS. 6A and 6B. The images result from phantom TPh (a) with no (0) lumps, showing no change in resistance; (b) with one (1) lump, showing one area with a resistance increase; and (iii) with two (2) lumps, showing two regions with a resistance increase. One lump is deeper in the tissue than the other, leading to a smaller signal change at that location. The pressure increases from zero in the first, leftmost column up to 80 mm Hg in the center column, and then the pressure decreases back down to 0 mm Hg in the last rightmost column in intervals of 20 mm Hg. The changes in resistance increase with pressure, as illustrated in FIG. 5B. EIT is described below with respect to FIGS. 11A and 11B as an example of a method and the associated hardware for imaging changes in conductivity of stretchable strain sensor 100.

FIG. 7B shows a close-up of the image 135″ in FIG. 7A taken at 80 mm Hg with phantom TPh containing two (2) lumps. The arrows show the positions of two peaks with areas APa and APb.

FIG. 7C shows a contour plot or image 135″a of the data in FIG. 7B. The arrows show the positions of the peaks, which have areas APa and APb, in this image.

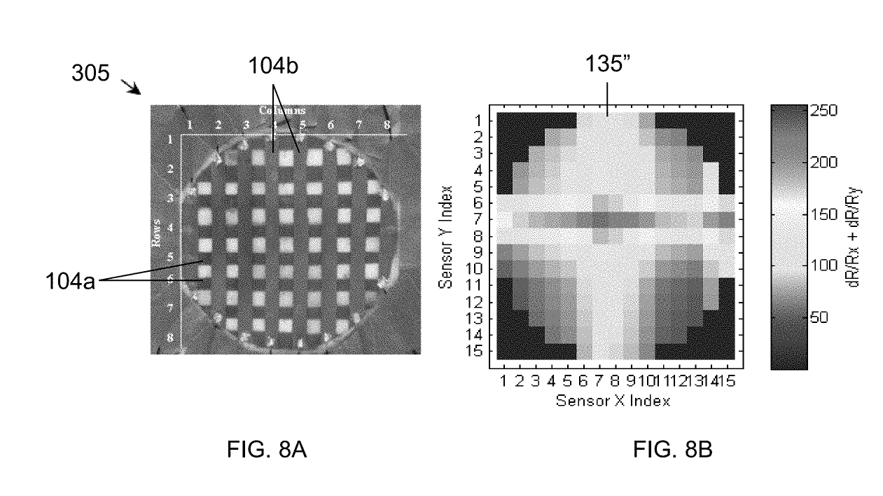

FIG. 8A illustrates an array 305 of strip-shaped sensors formed from a series of orthogonally positioned crossing strips of eight (8) rows 104a, numbered 1 to 8, and eight (8) columns 104b, numbered 1 to 8, as illustrated in FIG. 3E.

FIG. 8B illustrates the corresponding image 135″ in response to a stretch imposed at row 4, column 4.

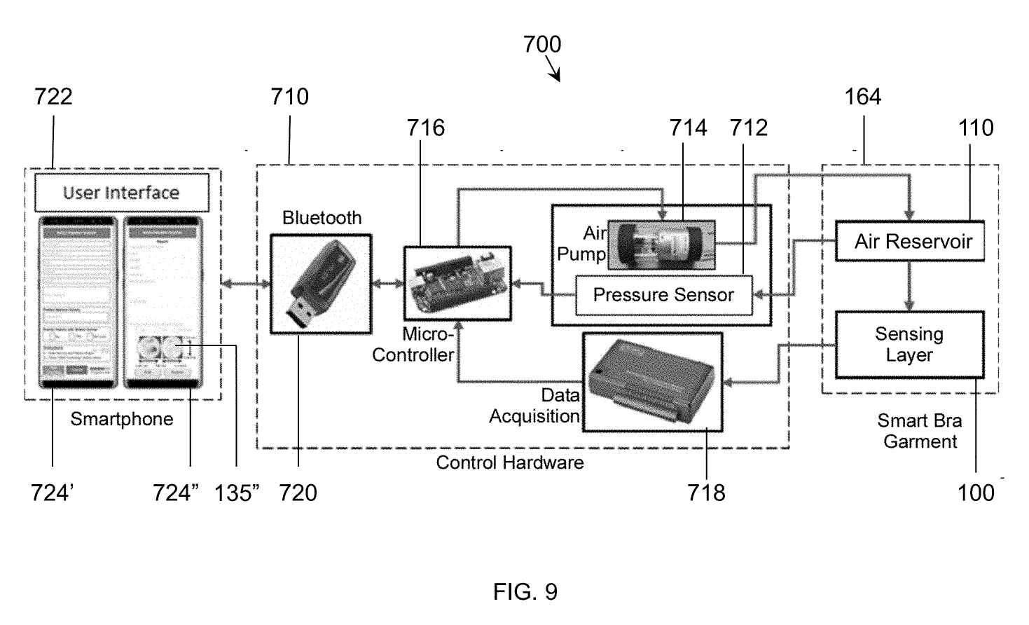

FIG. 9 illustrates an embodiment of a tumor detection system 700 that includes a mechatronic system 710 including a micro-controller 716 for storing and executing instructions that enable mechatronic system 710 to: inflate reservoir 110 using pump 714 in order to apply pressure to an anatomical feature of the subject, and prior to or during or following inflation of reservoir 110 collect data from pressure sensor 712 and sensing layer 100, utilizing data acquisition system 718 and microcontroller 716, and transmit the data to a computing device 722 using a transmitting module 720. Computing device 722 transmits instructions obtained from a user via graphical interface screen 724′ to microcontroller 716, performs calculations on data received from microcontroller 716 to create an image 135″, and displays image 135″ on graphical user interface screen 724″. Subsystems 710 and 722 can create image 135″ from any one of the tactile sensing systems 10, 15, 20, 30, 50, 60, 160, 162, 164,166, or 167a or 167b.

Within the smart bra garment 164 are a stretchable strain sensing layer 100 and an inflatable reservoir or air bladder 110. Reservoir 110 is inflated using an air pump 714 and its pressure is read by a pressure sensor 712. Anticipated pressures are in the range of 100 mm Hg. Strain sensing layer 100 is in electrical communication with a data acquisition system, DAQ 718. Pressure sensor 712, DAQ 718, and pump 714 are in communication with a microcontroller 716. Microcontroller 716 is in wireless communication with a computing device 722, which is a smartphone receiving instructions on a screen 724′ and displaying information on a screen 724″.

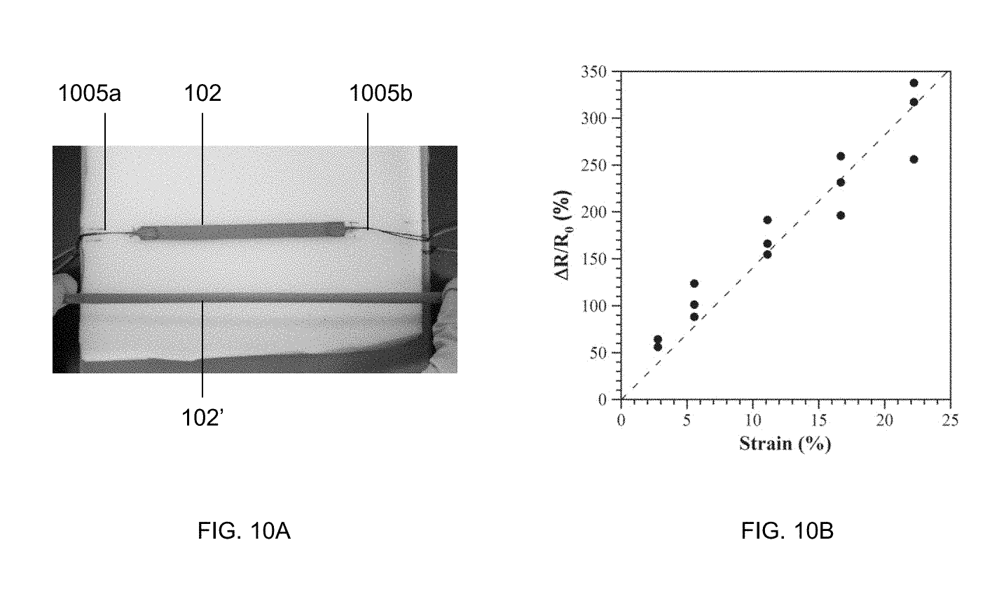

FIG. 10A illustrates an embodiment of strip-shaped sensors, as illustrated in FIG. 3B, in the unstretched state 102 and the stretched state 102′. Electrical leads 1005a and 1005b are attached to either end of strips 102 and 102′.

FIG. 10B illustrates the relative change in resistance on the Y-axis, ΔR/R₀, as a function of strain on the X-axis (%), for a strip-shaped sensor such as sensor 102, where ΔR is the change in resistance and R₀ is the original resistance in the unstretched state. The slope of the line is the gauge factor GF, which is the sensitivity of the sensor. In this embodiment, the GF is constant as a function of strain, since the slope is a straight line.

FIG. 11A illustrates a system 500 for performing electrical impedance tomography (EIT) on a continuous sensor 501 to reveal the amplitudes and positions of changes in the resistance of sensor 501, which is the same as continuous sensor 101 shown in FIG. 3A or continuous sensor 100 in FIGS. 5F and 5G, except in FIG. 11A, sensor 501 has a circular periphery as compared to continuous sensor 101 shown in FIG. 3A. The hardware to collect EIT data includes a controller 540 having an analog to digital converter 542 and digital output channels 543, multiplexers 530a and 530b to change the points 502 at which currents I, 520, are input and voltages V, 510, are measured. In this embodiment, there are 16 electrical connections 502₁-502₁₆ that serve as current injection electrodes and voltage measurement electrodes. Multiplexer 1, 530a, and multiplexer 2, 530b, are connected to each of the electrodes 502₁-502₁₆ by individual leads as shown in FIG. 6C. For simplicity, in FIG. 11A, only the current lead 1005_{I}, ground lead 1005_{G}, and voltage lead 1005_{V} that are active during a particular measurement time are shown.

In the “adjacent” method of EIT, current source 520 injects current into a pair of adjacent electrodes 502_{I} and 502_{G} via multiplexer 1, 530a. Via multiplexer 530b voltage 510 between electrodes 502_{V} and 502G is measured at every electrode except 502_{I} and 502_{G} to determine the voltages between all other pairs of adjacent electrodes. As shown in FIG. 11A, multiplexer 530b switches among electrodes 502₁-502₁₆ around the perimeter 505 of sensor 501 to read the voltages. Then multiplexer 530a switches the current injection pair position, rotating it by one position. Following that, the voltage measurement pattern is swept through the remaining positions by multiplexer 530b. The process continues until all 16 current injection positions have been employed. In this example, 502_{G} is at 502₁, 502_{I} is at 502₂, and 502_{V} is at 502₁₄. In the next step, 502_{V} will be at 502₁₅; in the next 502_{V} will be at 502₁₆; Thereafter, 502_{G} moves to 502₂, 502_{I} moves to 502₃, and 502_{V} moves to 502₄, then 502₅, etc., sweeping over the electrodes until reaching 502₁. At that point the current-injecting electrodes 502_{I} and 502_{G} will move over one more position, voltages will be read sequentially over the other electrodes to which current is being injected, and this process repeats until the current-injecting electrodes reach positions 502₁and 502₁₆. Data taken at subsequent time periods, for example at different pressures, are compared using an algorithm to show changes in surface conductivity between those times.

FIG. 11B illustrates the process for obtaining the strain distribution from the measured boundary voltages 550. The EIT software, for example the open source code EIDORS, solves the inverse problem in step 560 to obtain the conductivity distribution 570″. Conductivity distribution 570″ is then converted in step 580, using the gauge factor GF, into the strain distribution 590. EIT is merely one example of systems and methods which may be employed to measure changes in resistance of sensor 501.

EIT can be applied to other shapes of continuous sensors, such as squares, etc. Rather than using the EIT algorithm, machine learning could alternatively be used to determine the amplitudes and positions of changes in conductivity based on a set of training data, as is known to those in the art.

For strip-shaped sensors such as shown in FIG. 3B and FIG. 3C, two-point measurements are typically employed, such as shown in FIG. 10A. This will reveal the amplitude of a resistance change, but not its position along the strip.

An alternative method of obtaining position is to use multiple strip-shaped sensors, as shown in FIG. 3D or FIG. 3E. Use of the array of strips in FIG. 3D will give the position of a change in resistance in the vertical direction, perpendicular to the strips. Use of the array of strips in FIG. 3E will give the position of a change in resistance in both the vertical and horizontal directions. Yet another method for obtaining position is to use an array of “point” sensors, typically connected at two points, such as shown in FIG. 10A. Since the position of each point sensor along X and Y is known a priori, the location of a change in resistance of any of the point sensors reveals the position of a local strain.

Other systems and methods include machine learning or deep learning. Data for machine learning or deep learning may include electrical data such as voltage, current, resistance, impedance, inductance, etc.; optical data such as light intensity or phase; or acoustical data such as sound intensity or phase. Any such data that is not electrical is generally converted to electrical data and may be maintained in analog form or converted to digital form. The machine learning or deep learning may also include combinations of electrical data, optical data, or acoustical data.

As can be appreciated from the foregoing description of FIGS. 6A-11B, and referring most particularly to FIG. 9, the present disclosure relates also to a computational system, e.g., tumor detection system 700, that includes at least one computing device, e.g., in FIG. 9 microcontroller 716 and system 722, including a processor and a non-transitory memory storing instructions which, when executed by the processor of microcontroller 716 or system 722, cause the computing device to, following inflation of at least one inflatable reservoir 110a-110e, which may be effected in conjunction with an anatomical contact structure:
a) collect data from stretchable strain sensing layer 100 or 1001 via data acquisition system (DAQ) 718 and microcontroller 716; and
b) create an image, e.g., plot or image 135″ in FIG. 7B, from the data indicative of the amplitude and location of indentations or protrusions of at least one stretchable strain sensing layer 100-107, 501, or 1001 or display an image from the tactile sensing system, e.g., such as systems 10 to 30 in FIGS. 1A-1J, which may employ other strain sensing layers such as, for example, those illustrated and described with respect to FIGS. 3A-5I, relating to the data collected from the tactile sensing system.

The data acquired by computational system 700 may enable concluding that there is a mass or a void having a stiffness different from the surrounding tissue within the anatomical feature of a subject.

The data acquired by computational system 700 may enable concluding that there is an absence of least one mass having a stiffness different from the surrounding tissue within the anatomical feature of a subject.

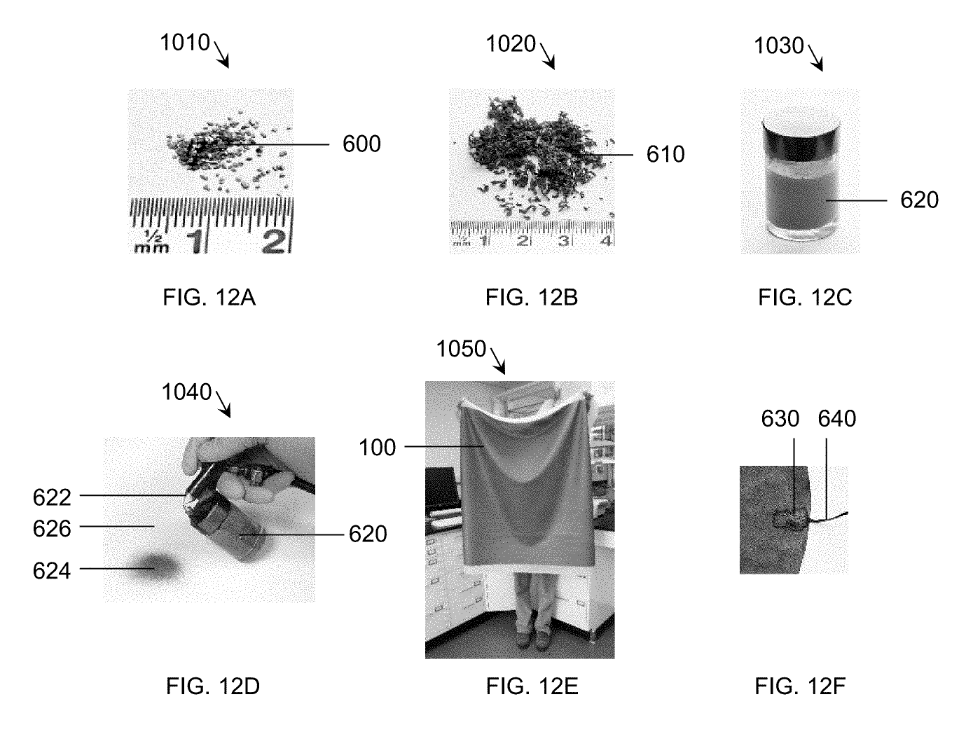

FIGS. 12A-12F illustrate a method of manufacturing strain sensing layer 100 as a piezoresistive latex/EG film coated onto a rubber membrane which includes the following steps.

FIG. 12A shows step 1010 of providing acid-intercalated graphite 600.

FIG. 12B shows step 1020 of providing expanded graphite 610 after heating graphite 600, for example in a microwave oven, the volume of each particle having expanded many fold.

FIG. 12C shows step 1030 of providing a suspension of exfoliated graphite (EG) and latex in water, the EG having been obtained by sonicating expanded graphite 610 in an aqueous solution to separate the layers.

FIG. 12D shows step 1040 of spray-coating an aqueous suspension 620 of EG and latex onto the surface of a latex membrane 626 using a sprayer 622 to form a thin film of latex/EG 624.

FIG. 12E shows the step 1050 of providing a strain sensing layer 100, where strain sensing layer 100 is formed from a latex membrane onto which has been spray coated a latex/EG layer.

FIG. 12F shows a carbon fiber yarn 640 serving as an electrical lead attached to a thin film of latex/EG coated onto a latex membrane using droplets of latex/EG 630.

The tactile system may be applied, for example by a robot, to tactile detection of protrusions. The protrusions may include bumps on an object, the edge of an object, or touch by a finger, among other things.

When configured as a tumor detection system, part of the anatomical support structure may comprise a surface of the inflation reservoir, and the stretchable strain sensing layer may comprise a surface of the inflation reservoir, as illustrated in FIG. 4B. Alternatively, the inflatable reservoir may be a stand-alone air bladder, for example such as is used in a blood pressure measuring cuff or for example a balloon, as illustrated in FIG. 4A. One or both of the strain sensing layer and the anatomical support structure may be integrated into the inflatable reservoir.

The system may be applied to non-anatomical masses and at least to anatomical masses in general, i.e. not just those which extend from the body, for example, measuring for lumps in the abdomen or on a limb.

The tumor detection system is thus also capable of detecting masses containing other biological or non-biological materials beyond the definition of “tumor”.

As indicated above, additional body surfaces and conditions other than tumors may be measured such as the limbs or torso, whether in males or females, e.g., cysts.

An inflatable reservoir is positioned over a tissue surface whereby inflation leads to a deformation of the tissue. At least one stretchable strain sensing layer is positioned whereby the deformation of a tissue containing a hard mass leads to a strain in that layer. This enables detection of a tumor or other anatomical structure within the tissue by the tumor detection system.

A smart bra embodiment of the system may include a piezoresistive sensing layer and an inflatable balloon built into a fabric bra, along with a portable electronic system.

As can be appreciated from the foregoing, the present disclosure relates to a method for performing automated palpation that includes the steps of:

(a) placing at least one stretchable strain sensing layer, e.g., sensing layers 100 to 107 or any others described above and illustrated in the figures, in contact with a region of an anatomical feature of a subject, e.g., breast b1 and/or breast b2;
(b) applying pressure to at least a portion of the anatomical feature of a subject using an inflatable reservoir, e.g., reservoirs 110, 111 in conjunction with an anatomical contact structure, e.g., anatomical contact structure 210; and
(c) detecting signals from the stretchable strain sensing layer,
wherein a signal results from the presence of a mass M having a stiffness different from surrounding tissue T within the anatomical feature of a subject.

The applying of the pressure may include increasing the pressure from zero to a maximum value and acquiring measurements at intervals of the pressure.

While several embodiments and methodologies of the present disclosure have been described and shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular embodiments and methodologies. Those skilled in the art will envision other modifications within the scope of the claims appended hereto.

What is claimed is:

1. A tactile sensing system comprising:
at least one stretchable strain sensing layer;
at least one inflatable reservoir; and
an anatomical contact structure configured to enable the at least one stretchable strain sensing layer to be in contact with a region of an anatomical feature of the subject and
configured to enable the at least one inflatable reservoir to apply pressure to a region of the anatomical feature of the subject.

2. The tactile sensing system according to claim 1, wherein at least a portion of the region to which pressure is enabled to be applied by the at least one inflatable reservoir at least partially corresponds to the portion of the region with which the at least one stretchable strain sensing layer is in contact.

3. The tactile sensing system according to claim 1, wherein the region of an anatomical feature of the subject to which pressure is enabled to be applied by the at least one inflatable reservoir does not correspond to the region of an anatomical feature of the subject with which the at least one stretchable strain sensing layer is in contact.

4. The tactile sensing system according to claim 1, wherein the tactile sensing system is configured wherein inflation of the at least one inflatable reservoir to apply pressure to a region enables detection by the at least one stretchable strain sensing layer of at least one mass having a stiffness different from surrounding tissue within the anatomical feature feature of a subject.

5. The tactile sensing system according to claim 1, wherein the tactile sensing system is configured wherein inflation of the at least one inflatable reservoir to apply pressure to a region enables concluding via the at least one stretchable strain sensing layer of the absence of least one mass having a stiffness different from the surrounding tissue within the anatomical feature feature of a subject.

6. The tactile sensing system according to claim 1, wherein the at least one stretchable strain sensing layer is configured to be disposed in contact with the at least one inflatable reservoir, enabling thereby:
formation of an indentation in the at least one inflatable reservoir and localized strain in the at least one stretchable strain sensing layer around the indentation in the at least one inflatable reservoir.

7. The tactile sensing system according to claim 1 wherein the tactile sensing system comprises:
at least one stretchable strain sensing layer configured and disposed to enable contact with a first region of an anatomical feature of a subject;
at least two inflatable reservoirs configured and disposed to enable the at least two inflatable reservoirs to apply pressure to a second and third region of an anatomical feature of a subject,
the at least two inflatable reservoirs configured and disposed to be independently inflatable with respect to one another such that one of the at least two inflatable reservoirs is enabled to apply an initial pressure to the second region of the anatomical feature of a subject that is greater than the pressure applied to the third region of the anatomical feature of a subject by the at least another one of the at least two inflatable reservoirs,
the at least two inflatable reservoirs configured and disposed such that the at least another of the at least two inflatable reservoirs is enabled to apply a pressure to the third region of the anatomical feature of a subject following or during deflation of the initial pressure applied to the second region of the anatomical feature of a subject by the one of the at least two inflatable reservoirs,
enabling thereby detection by the at least one stretchable strain sensing layer of at least one mass having a stiffness different from the surrounding tissue within the anatomical feature of a subject.

8. The tactile sensing system according to claim 7, wherein
yet another one of the at least two inflatable reservoirs is configured and disposed such that the yet another of the at least two inflatable reservoirs is enabled to apply a pressure to a fourth region of the anatomical feature of a subject following deflation of the initial pressure applied to the second region of the at least one anatomical feature of a subject by the one of the at least two inflatable reservoirs and following deflation of the initial pressure applied to the third region of the at least one anatomical feature of a subject by the at least another one of the at least two inflatable reservoirs,
enabling thereby detection by the at least one stretchable strain sensing layer of at least one mass having a stiffness different from surrounding tissue within the anatomical feature of a subject.

9. The tactile sensing system according to claim 8, wherein the tactile sensing system is configured and disposed to enable each of the at least two inflatable reservoirs to be inflated and deflated sequentially in a pattern imitating manual palpation of an anatomical feature of a subject.

10. The tactile sensing system according to claim 1 wherein the tactile sensing system is configured and disposed to enable increasing the pressure from zero to a maximum value and acquiring measurements at intervals of the pressure.

11. The tactile sensing system according to claim 1, wherein the at least one stretchable strain sensing layer includes at least one continuous sensor or at least one array of stretchable strain sensing layers or a combination of at least one continuous sensor and at least one array of stretchable strain sensing layers,
enabling thereby the formation of an image indicative of location of at least one mass in the anatomical feature of a subject.

12. The tactile sensing system according to claim 11 wherein the tactile sensing system is configured to enable injection of currents and the reading of voltages at selectable portions of the at least one stretchable strain sensing layer, wherein the at least one stretchable strain sensing layer is a continuous strain sensor.

13. The tactile sensing system according to claim 11 wherein the formation of an image is enabled by configuring the tactile sensing system to utilize one of electrical impedance tomography and machine learning.

14. The tactile sensing system according to claim 13, wherein the machine learning includes utilization of one of electrical data or optical data or acoustical data or combinations thereof.

15. The tactile sensing system according to claim 1, wherein the anatomical contact structure comprises a cup-shaped structure.

16. The tactile sensing system according to claim 15 wherein the cup-shaped structure is one of the cup of a brassiere and a male athletic supporter.

17. The tactile sensing system according to claim 1, wherein
the at least one stretchable sensing layer includes a first stretchable sensing layer and a second stretchable sensing layer that are spaced apart from one another,
each of the first stretchable strain sensing layer and the second stretchable sensing layer configured to be disposed in contact with at least one anatomical feature of a subject; and
at least one inflatable reservoir configured and disposed to enable application of pressure to at least one region of an anatomical feature of a subject,
enabling thereby the location of at least one mass in the anatomical feature of a subject.

18. The tactile sensing system according to claim 1, comprising a computational system, the computational system comprising:
a computing device including a processor and a non-transitory memory storing instructions which, when executed by the processor, cause the computing device to, prior to or during or following inflation of the at least one inflatable reservoir:
collect data from the at least one stretchable strain sensing layer; and
create an image from the data indicative of the amplitude and location of indentations of the at least one stretchable strain sensing layer.

19. The tactile sensing system according to claim 1, wherein the stretchable strain sensing layer is piezoresistive.

20. A system for examining an anatomical feature of a subject comprising:
a computing device including a processor and a non-transitory memory storing instructions which, when executed by the processor, cause the computing device to:
prior to or during or following inflation of at least one inflatable reservoir to apply pressure to at least one anatomical feature of a subject;
collect data from a stretchable tactile sensor in contact with the at least one anatomical feature of the subject; and
display an image relating to the data collected from the sensor that indicates the spatial locations and magnitudes of local strains in the sensor.

21. A tactile sensing system comprising:
at least one stretchable strain sensing layer;
at least one inflatable reservoir,
the at least one stretchable strain sensing layer configured to be disposed in contact with the at least one inflatable reservoir enabling thereby:
indentation of the at least one inflatable reservoir and localized strain in the at least one stretchable strain sensing layer around an indentation of the at least one inflatable reservoir; and
a computational system comprising:
a computing device including a processor and a non-transitory memory storing instructions which, when executed by the processor, cause the computing device to, prior to or during or following inflation of the at least one inflatable reservoir:
collect data from the at least one stretchable strain sensing layer; and
create an image from the data indicative of the amplitude and location of indentations of the at least one stretchable strain sensing layer,
wherein strain in the at least one stretchable strain sensing layer is caused by touch by an object or being.
